(19) United States
(12) Patent Application Publication
(10) Pub. No.: US 2003/0053999 A1
(43) Pub. Date: Mar. 20, 2003

(54) Enzyme with xylanase activity

(76) Inventors: Jean-Luc Jonniaux, Tienen (BE); Thierry Dauvrin, Couthuin (BE)

Correspondence Address: KNOBBE MARTENS OLSON & BEAR LLP, 2040 MAIN STREET, FOURTEENTH FLOOR, IRVINE, CA, 92614, US

(21) Appl. No.: 09/790,070

(22) Filed: Feb. 21, 2001

(30) Foreign Application Priority Data
Feb. 21, 2000 (EP) 00870028.8

(51) Int. Cl. A61K 38/46; C12N 9/24; C12P 21/02; C12N 1/21; C12N 15/74; C07H 21/04

(52) U.S. Cl. 424/94.6; 435/252.3; 435/69.1; 435/320.1; 435/200; 536/23.2

(57) ABSTRACT

The present invention is related to an isolated and purified enzyme with xylanolytic activity having more than 70% homology with the amino acid sequence SEQ ID NO 11.

### FIELD OF THE INVENTION

The present invention relates to an enzyme with xylanase activity identified by its amino acid and nucleotide sequences and variants thereof.

The present invention relates also to their uses in the agrofood and in the pulp and paper industries.

### BACKGROUND OF THE INVENTION

Xylans are heteropolysaccharides which form the major part of the hemicellulose present in the plant biomass.

The backbone of these polysaccharides is a chain of β-1,4 linked xylopyranosyl residues. Many different side groups could bind to these residues like acetyl, arabinosyl and glucuronosyl residues. Phenolic compounds such as ferulic or hydroxycinnamic acids are also involved through ester binding in the cross linking of the xylan chains or in the linkage between xylan and lignin chains for example.

Endoxylanases hydrolyze specifically the backbone of the hemicellulose. In some cases, the side groups may mask the main chain by steric hindrance. Different xylanase activities already described are characterized by their specificity towards their substrate and the length of the oligomers produced.

These differences between the xylanases concerning their properties seem to be partly related to their respective amino acid sequences. Endoxylanases have been classified into two families (F or 10 and G or 11) according to their sequence similarities (Henrissat & Bairoch, (1993),Biochem. J., vol. 293, p. 781.). The F family of xylanases are larger, more complex as compared to the G family of xylanases. Moreover the F family xylanases produce small oligosaccharides, while the G family xylanases show a higher affinity for unsubstituted xylan.

Xylanases are used in various industrial areas such as the pulp, paper, feed and bakery industries. Other applications include the juice and beer industries. Xylanases could also be used in the wheat separation process. The observed technological effects are, among others, improved bleachability of the pulp, decreased viscosity of the feed or changes in dough characteristics.

Many different microbial genera have been described to produce one or several xylanases. These microbial genera comprise bacteria as well as eukaryotic organisms like yeast or fungi.

### SUMMARY OF THE INVENTION

The present invention relates to providing an isolated and purified enzyme with xylanase activity.

Another aspect of the present invention provides a method for using the enzyme with xylanase activity in different kinds of industries such as agrofood, pulp, paper industries.

### BRIEF DESCRIPTION OF THE DRAWINGS

FIG. 1 shows a SDS-polyacrylamide gel of the proteins recovered after the successive purification steps of the enzyme with xylanolytic activity.

FIG. 2 shows a Southern blot analysis of the Penicillium griseofulvum A160 genomic DNA.

FIG. 3 represents the complete genetic sequence of the xylanase according to the invention.

FIG. 4 shows the effect of the temperature and of the pH on the xylanase activity.

FIG. 5 represents the increase of a bread volume according to the enzymatic activity of the xylanase according to the invention.

### DETAILED DESCRIPTION OF THE PREFERRED EMBODIMENT

A first aspect of the present invention is related to an isolated and purified (from possible contaminants) xylanase amino acid sequence presenting more than 50%, preferably more than 70, 80 or 85%, more preferably more than 90% homology (or sequence identity) with the amino acid sequence SEQ ID NO 11.

Advantageously, the isolated and purified xylanase amino acid sequence according to the invention has a molecular weight comprised between 22 kD and 26 kD, preferably a molecular weight approximately 24 kD.

Said xylanase amino acid sequence or peptide is expressed extracellularly or intracellularly and/or secreted by the recombinant host cell according to the invention.

According to another preferred embodiment of the present invention, the isolated and purified xylanase amino acid sequence has the amino acid sequence of SEQ ID NO 11 or a smaller portion of said amino acid sequence (of more than 30 or 50 amino acids, preferably more than 100 amino acids), which has at least more than 80% of the xylanase activity of the complete amino acid sequence SEQ ID NO 11, preferably more than 95% of the xylanase activity or the complete xylanase activity of the complete amino acid sequence SEQ ID NO 11 (see also Example 1). In other words, the isolated and purified xylanase amino acid sequence according to the invention may be deleted partially while maintaining its enzymatic activity, which may be measured by methods well known by persons skilled in the art.

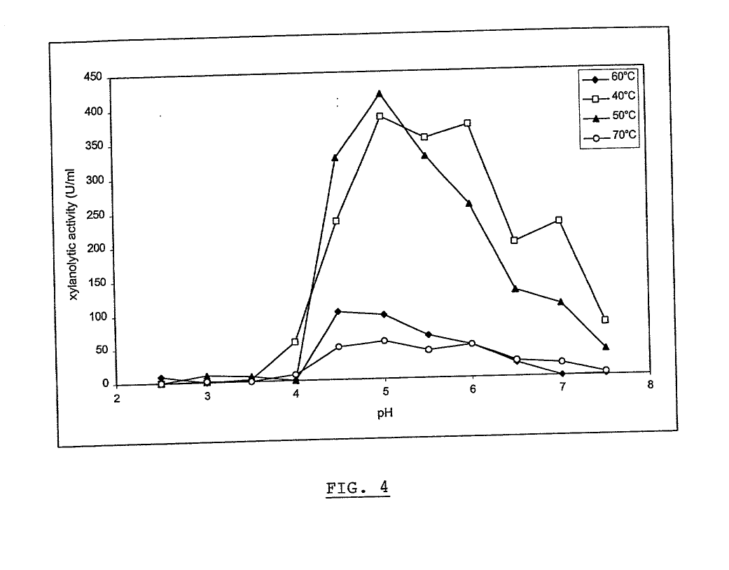

The purified xylanase enzyme according to the invention is also characterized by an optimum pH around pH 5.0 and temperature profile having its maximum activity at about 50° C. More generally, the maximum activity of the enzyme is between pH 4.5 and 7.0, at a temperature comprised 35° C. and 55° C. (see FIG. 4).

The present invention is also related to an isolated and purified nucleotide sequence from a microorganism, encoding a xylanase. Preferably, said microorganism is selected from the group consisting of bacteria or fungi (including yeast), preferably the Penicillium species fungi, more specifically Penicillium griseofulvum.

According to a preferred embodiment of the present invention, said microorganism is Penicillium griseofulvum having the deposit number MUCL-41920.

According to the invention, said nucleotide sequence presents more than 50%, preferably more than 70%, more preferably more than 90% homology (or sequence identity) with the sequence SEQ ID NO 8 described hereafter.

According to a preferred embodiment of the present invention, said isolated and purified nucleotide sequence corresponds to the nucleotide sequence SEQ ID NO 8 or a portion thereof encoding a peptide having a xylanase activity.

It is meant by “a portion of the nucleotide sequence SEQ ID NO 8”, a fragment of said sequence SEQ ID NO 8 having more than 90 nucleotides, preferably more than 100 nucleotides or more than 120 nucleotides, of said nucleotide sequence and encoding a protein characterized by a xylanase enzymatic activity similar to the xylanase activity of the complete amino acid sequence SEQ ID NO 11. Preferably, said portion has a xylanase enzymatic activity of more than 80% of the initial xylanase enzymatic activity of the complete enzyme defined by its amino acid sequence SEQ ID NO 11, preferably has a xylanase enzymatic activity corresponding to the one of amino acid sequence SEQ ID NO 11.

Another aspect of the present invention is related to a recombinant nucleotide sequence comprising, operably linked to the nucleotide sequence according to the invention and above-described, one or more adjacent regulatory sequence(s), preferably originating from homologous microorganisms. However, said adjacent regulatory sequences may also be originating from heterologous microorganisms. These adjacent regulatory sequences are specific sequences such as promoters, secretion signal sequences and terminators.

Another aspect of the present invention is related to the vector comprising the nucleotide sequence(s) according to the invention, possibly operably linked to one or more adjacent regulatory sequence(s) originating from homologous or from hetereologous microorganisms.

It is meant by “a vector”, any biochemical construct which may be used for the introduction of a nucleotide sequence (by transduction, transfection, transformation, infection, conjugation, etc.) into a cell. Advantageously, the vector according to the invention is selected from the group consisting of plasmids, viruses, phagemids, chromosomes, transposons, liposomes, cationic vesicles or a mixture thereof. Said vector may comprise already one or more of the above-described adjacent regulatory sequence(s) (able to allow its expression and its transcription into a corresponding peptide by said microorganism). Preferably, said vector is a plasmid incorporated into E. coli and having the deposit number LMBP-39987.

The present invention is also related to the host cell, preferably a recombinant host cell, “transformed” by the nucleotide sequence or the vector according to the invention above-described.

It is meant by “a host cell “transformed” by the nucleotide sequence or the vector according to the invention”, a cell having incorporated said nucleotide sequence or said vector and which does not comprise naturally (originally) said nucleotide sequence. The transformed host cell may also comprise a cell having incorporated said vector or said nucleotide sequence by genetic transformation, preferably by homologous recombination or other method (recombinant microorganism).

A “host cell” may be also the original cell comprising the nucleotide sequence encoding the enzyme according to the invention and genetically modified (recombinant host cell) to overexpress or express more efficiently said enzyme (better pH profile, higher extracellular expression, etc.).

Preferably, said host cell is also capable of overexpressing (higher expression than the expression observed in the initial microorganism) said nucleotide sequence or said vector and allows advantageously a high production of an amino acid sequence encoded by said nucleotide sequence or by said vector. The isolated and purified nucleotide sequence according to the invention may be either integrated into the genome of the selected host cell or present on an episomal vector in said host cell.

Advantageously, the recombinant host cell according to the invention is selected from the group consisting of the microbial world, preferably bacteria or fungi (including yeast).

Preferably, said recombinant host cell is modified to obtain an expression of the xylanase enzyme at a high level, obtained by the use of adjacent regulatory sequences being capable of directing the overexpression of the nucleotide sequence according to the invention in the recombinant host cell or by increasing the number of nucleotide copies of the sequences according to the invention.

The following description describes also the conditions (culture media, temperature, pH conditions, etc.) for the culture of the host selected for the expression of the xylanase according to the invention. For this purpose, the original production species and/or a suitable host cell transformed with a DNA construct designed to express the said enzyme are present in a suitable growth medium.

According to the present invention, said protein with xylanolytic activity may be isolated from the medium and/or purified. The culture, isolation and purification conditions are derived from conventional methods well-known to persons skilled in the art.

The xylanase enzyme according to the invention may be used in different kinds of industries.

The enzyme with xylanolytic activity of the present invention, purified or not purified, is particularly suited as a bread-improving agent. Bread-improving agents are products which could improve or increase texture, flavor, anti-staling effect, softness, crumb softness upon storage, freshness and machinability, volume of a dough and/or of a final baked product. Preferably, said enzyme with xylanolytic activity increases the specific volume of the final baked product.

“Baked product” intends to include any product prepared from dough, in particular a bread product. Dough is obtained from any type of flour or meal (for example, based on rye, barley, oat or maize), preferably prepared with wheat or with mixes including wheat.

A further aspect of the present invention relates to the additive effect of said enzyme having xylanolytic activity with other enzymes, in particular with an alpha-amylase, preferably an alpha-amylase from Aspergillus oryzae. Said enzyme with xylanolytic activity may be used in combination with other bread-improving agents like enzymes, emulsifiers, oxidants, milk powder, fats, sugars, amino acids, salts, or proteins (gluten, cellulose binding site) well known to persons skilled in the art.

According to the present invention, the enzyme with xylanolytic activity, purified or not, shows hydrolytic activities in presence of plant cell wall components. Particularly, said enzyme degrades the wheat cell wall components. Particularly, the degradation activities lead to a decrease of the flour viscosity in the presence of water. Said enzyme may thus advantageously be used in the separation of components of plant cell materials such as cereal components. Particularly, said enzyme may be used to improve the separation of the wheat into gluten and starch by the so-called batter process.

According to the present invention, said enzyme may be used to improve the filtrability and/or decrease the viscosity of glucose syrups obtained from impure cereal starch by subjecting the impure starch first to the action of an alpha-amylase, then to the action of said xylanase. It may also be used in beer brewing when cereal has to be degraded to improve the filtrability of the wort or to reuse the residuals from beer production for example, animal feed. Said enzyme may be used in feed to improve the growth rate or the feed conversion ratio of animals such as poultry.

Another application resides in the oil extraction where oil has to be extracted from the plant material such as the corn oil from corn embryos. The enzyme with xylanolytic activity of the present invention may be used in fruit and vegetable juice processing to improve the yield. According to the present invention, said enzyme may be used in all processes involving plant materials or waste materials, for example, from paper production, or agricultural wastes such as wheat-straw, corn cobs, whole corn plants, nut shells, grass, vegetable hulls, bean hulls, spent grains, sugar beet, and the like.

The effect of the enzyme with xylanolytic activity of the present invention may be further improved by adding other enzymes in combination with said enzyme. Such enzymes may belong, but are not restricted to, hydrolytic enzymes families such as glucanases, proteases, cellulases, hemicellulases, or pectinases. Other enzymes are transglutaminases, oxido-reductases and isomerases, etc.

The enzyme with xylanolytic activity according to the invention may be used under several forms. Cells expressing the enzyme, such as yeast, fungi, archaea or bacteria, may be used directly in the process. Said enzyme may be used as a cell extract, a cell-free extract (i.e. portions of the host cell that have been submitted to one or more disruption, centrifugation and/or extraction steps) or as a purified protein. Any of the above-described forms may be used in combination with one or more other enzyme(s) under any of the above-described forms. These whole cells, cell extracts, cell-free extracts or purified enzymes may be immobilized by any conventional means on a solid support to allow protection of the enzyme, continuous hydrolysis of substrate and/or recycling of the enzymatic preparation. Said cells, cell extracts, cell-free extracts or enzymes may be mixed with different ingredients (such as in the form of a dry power or a granulate, in particular a nondusting granulate, in a form of a liquid, for example with stabilizers such as polyols, sugars, organic acids, sugar alcohols according to well-established methods).

The invention will be described in further detail in the following examples by reference to the enclosed drawings, without limiting its scope.

### EXAMPLES

### Example 1

### Purification of an Enzyme with Xylanolytic Activity from Penicillium griseofulvum A160

Strain

5 g of a commercial Belgian wheat flour were suspended in 50 ml saline solution (NaCl 0.9%). Aliquots of 100 μl of this suspension were spread on AMAM plates (Aspergillus Minimum Agar Medium: glucose 1%, NaNO₃ 0.6%, KCl 7 mM, Kh₂PO₄ 11 mM, MgSO₄ 2 mM, ZNSO₄ 76 μM, H₃BO₃ 178 μM, MnCl₂ 25 μM, FeSO₄ 18 μM, CoCl₂ 7.1, μM CuSO₄, 6.4 μM, Na₂MoO₄ 6.2 μM, EDTA 174 μM, pH 6.5 (Pontecorvo et al. (1953) Adv. Genet. vol. 5, p. 142.) supplemented with 1.5% bacto-agar and 100 μg/ml ampicilline.

Among the strains that appeared on the plates after incubation at 30° C., a particular strain was isolated and identified as Penicillium griseofulvum with the isolation reference A160 (MUCL-41920).

Determination of the Xylanolytic Activity

The xylanolytic activity was determined by measuring the reducing sugars formed from the Beechwood xylan (Sigma). The reducing sugars were revealed with the 2,3-dinitrosalicylic acid (Bailey et al. (1992) J. Biotechnol. vol. 23, p. 257.). The reaction was carried out by 30° C. in a 100 mM acetate buffer at pH 4.5. The xylanolytic activity was expressed in μmole xylose/min.

For rapid identification of the enzyme, the xylanolytic activity was assayed using Azo-xylan (Megazyme) as substrate following the supplier instructions with the exception that the reaction was carried out at 35° C. in a 100 mM citrate-phosphate buffer at pH 6.0.

In this case, one xylanase unit was arbitrarily defined as the amount of enzyme required to increase the optical density by one unit at 595 nm in 10 min.

Purification of the Xylanolytic Enzyme

The strain of Penicillium griseofulvum A160 was cultivated in 2 liters of Aspergillus Minimal Medium pH 6.5 (Ponteverco et al., 1953.), supplemented with 1% xylan from oat spelt (Sigma) at 30° C. After 72 hours, the culture was filtered through a Miracloth filter (Calbiochem) to remove the mycelium. The filtrate was concentrated by ultrafiltration in a Pellicon device with a 10 kDa Biomax 10 cassette (Millipore) to a final volume of 170 ml. The concentrate was diluted 3 times to reach a final concentration of 50 mM in sodium acetate pH 4.2.

This solution was loaded at 2 ml/min on a Pharmacia XK16/20 column filled with 30 ml of the Bio-Rad Macro High S resin equilibrated in 50 mM sodium acetate pH 4.2. Proteins were eluted with a linear increasing NaCl gradient from 0 M to 0.6 M in 50 mM sodium acetate pH 4.2. Xylanase activity was determined in the eluted fractions. Active fractions were pooled and equilibrated in 1.2 M ammonium sulfate, 50 mM sodium acetate pH 5.0 in a final volume of 65 ml.

These were applied on a Phenyl Sepharose HP column (Pharmacia) and eluted at 2.5 ml/min with a 1.2 M to 0 M ammonium sulfate linear gradient in a 50 mM sodium acetate pH 5.0 buffer. Xylanase activity was determined in the eluted fractions. The xylanase activity was collected as one peak at 0.8 M ammonium sulfate.

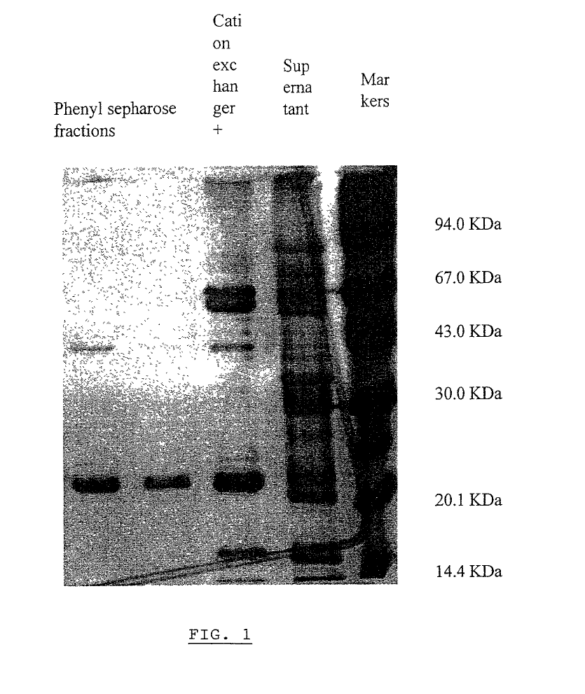

One major protein is present in this peak as shown by SDS-polyacrylamide gel (FIG. 1).

### Example 2

### Determination of the Amino Acid Sequence of the Enzyme with Xylanolytic Activity

General procedures were followed to perform the N-terminal sequencing of the protein after electrophoresis on a 12% SDS-polyacrylamide gel and electroblotting on a PVDF Immobilon-P membrane (Millipore). An automated 477A Protein Sequencer coupled to a HPLC 120A Analyzer (Applied Biosystems) was used.

The following sequence has been obtained with the protein with an apparent molecular weight of 24 kDa:

SEQ ID NO 1: D I T Q N E R G T N N G Y F Y S F W T X G G G N V Y

### Example 3

### Cloning of a Gene Coding for a Enzyme with Xylanolytic Activity

Cloning of Internal DNA Fragments

The genomic DNA from Penicillium griseofulvum A160 was isolated according to Boel et al. (EMBO J., vol. 7, p. 1581 (1984)). The strain was grown in 50 ml Aspergillus Minimum Medium supplemented with 0.5% Yeast Extract (Difco). After 24 hours, the mycelium was harvested by filtration on a Miracloth filter and washed twice with water. 1 g mycelium was incubated in 10 ml solution A (sorbitol 1 M, EDTA 25 mM, pH 8.0) for 30 min at 30° C. The cells were then centrifuged and suspended in 10 ml solution B (Novozym 234 20 mg, sorbitol 1 M, sodium citrate 0.1 M, EDTA 10 mM, pH 5.8). After 30 min at 30° C., the cells were centrifuged and lysed with 15 ml of solution C (phenol 40%, SDS 1%). DNA was separated from the contaminating material by successive extractions with phenol and phenol-chloroform, followed by ethanol precipitation.

The degenerate synthetic oligonucleotides mixtures SEQ ID NO 2 and SEQ ID NO 3 were designed based on the N-terminal sequence. A third synthetic oligonucleotides mixture SEQ ID NO 4 has been designed based on a hypothetical degenerate sequence coding for the amino acid sequence EYYIVD (SEQ ID NO 14), conserved among the family G xylanases.

| | |
|---|---|
| SEQ ID NO 2: | GGY TAY TTY TAY AAY TTY TGG AC |
| SEQ ID NO 3: | GGY TAY TAY TAY TCI TTY TGG AC |
| SEQ ID NO 4: | TCG ACR AYG TAG TAY TC |

In these sequences, Y stands for T or C, R for A or G, I for inosine.

The PCR reaction was carried out with 10 ng gDNA of Penicillium griseofulvum A160 in the presence of 5 pmole of each synthetic oligonucleotides mixture SEQ ID NO 2 and SEQ ID NO 3 and 10 pmole synthetic oligonucleotides mixture SEQ ID NO 4. The reaction mix contained also 1 unit rTAQ polymerase (Pharmacia), 200 μM dNTP, 50 mM KCl, 1.5 mM MgCl2 and 10 mM Tris-HCl pH 9.0 in a final volume of 25 μl. After 4 min of denaturation at 94° C., 25 cycles of [30 s 94° C., 30 s 50° C. and 45 s 72° C.] were performed followed by 7 min of elongation at 72° C. Only one fragment of 0.3 kb length was amplified as revealed by agarose gel electrophoresis.

1 μl of the PCR reaction described above was directly sequenced on a ABI 377 Sequencer (Applied Biosystems) with either 3 pmoles synthetic oligonucleotides mixtures SEQ ID NO 2 or SEQ ID NO 3 as primers. The sequencing with the oligonucleotides SEQ ID NO 2 and SEQ ID NO 3 gave the nucleotide sequences SEQ ID NO 5 and SEQ ID NO 6, respectively.

| | |
|---|---|
| SEQ ID NO 5: | CNAGTACAACAACGNNAAGNCCGGCNAATACAGNGTG |
| | NANTGGAAGAACTGCGGNTATTTCACCTCTGGCAAGG |
| | GCTGGANNACTGGTAGNGCCCGGTAAGT |
| SEQ ID NO 6: | CGGCNAATACAAGGGTGTNANTGGAAGAACTGCGGNN |
| | ATTTCACCTCNGGCAAGGGCTGGACTACTGGTAGTGC |
| | CCGGTAAGTGCAA |

A homology search with the above-mentioned sequences against the NCBI proteins database (Jan. 5, 1999) using the BLASTX 2.0.8 software found the best homology with the endo-β-1,4-xylanase A from Chaetomium (Accession number: dbj |BAA08649).

Southern Blotting of the Penicillium griseofulvum A160 Genomic DNA

Genomic DNA (0.5 μg) was digested overnight at 37° C. with either 2 units of the restriction enzyme EcoRI (Pharmacia), or 2 units each of restriction enzymes BamHI and EcoRI (Pharmacia), or 2 units of each restriction enzymes EcoRI and XbaI in a final volume of 20 μl (buffer: 1× One-Phor-All buffer PLUS (Pharmacia)). The digested DNAs were loaded on a 0.8% agarose gel in 1× TBE buffer. After electrophoresis, the restricted fragments were transferred onto a Hybond-N+ membrane (Amersham). The PCR fragments described above (1 μl) were labeled with digoxigenin using the DIG High Prime DNA Labeling and Detection Starter Kit II (Boehringer Mannheim). The membrane was hybridized overnight at 42° C. in the presence of a standard hybridization buffer (SSC 5×, formamide 50%, N-lauroylsarcosine 0.1%, SDS 0.02%, Blocking reagent) and a probe concentration of approximately 10 ng/ml (denatured for 5 min at 97° C.). After the hybridization, the membrane was first washed at 55° C. with 2× SSC, 0.1% SDS (2×15 min) followed by 3 washes with a 0.5× SSC, 0.1% SDS solution (30 min). After immunological detection, the hybridizing bands were identified by a four hour exposure to Kodak X-OMAT AR film at room temperature.

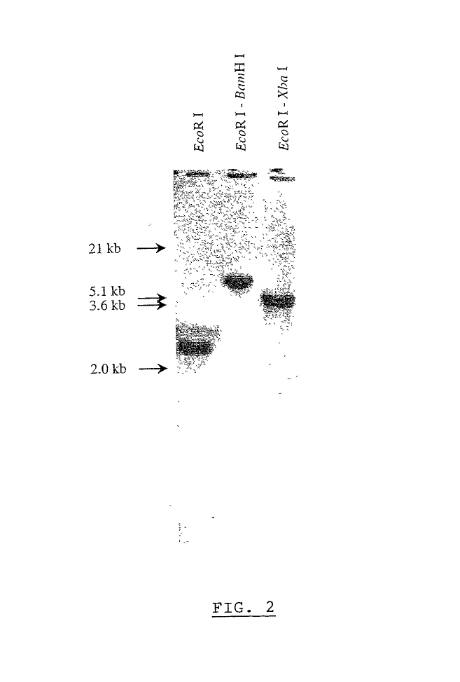

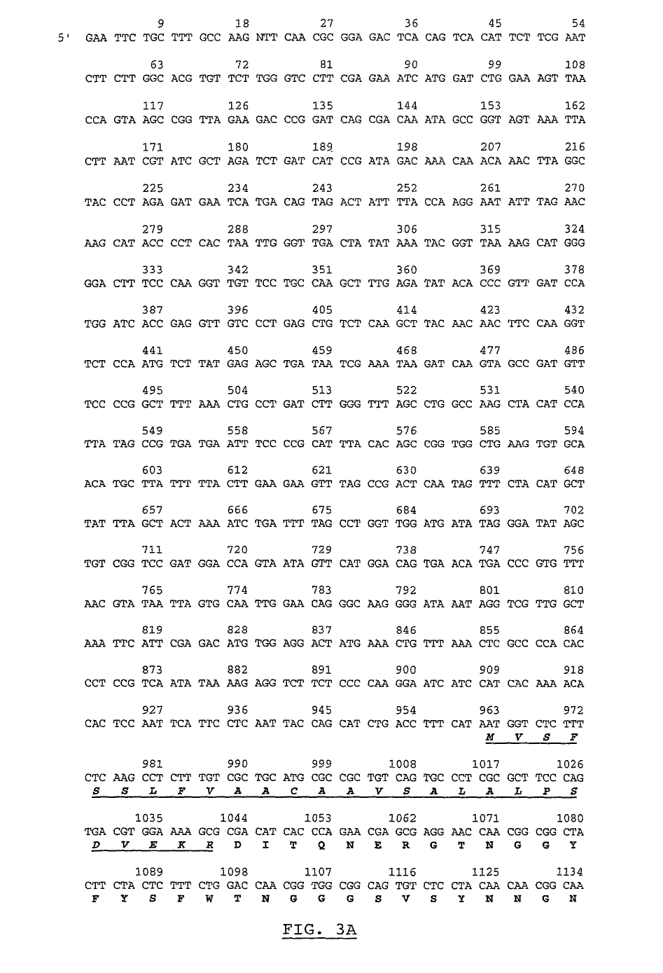

The results of the hybridization experiment are shown on the FIG. 2. It revealed that under the hybridization conditions tested, one DNA fragment hybridized with the probe.

Construction of a gDNA Restriction Fragments Library of Penicillium griseofulvum A160

Genomic DNA (5 μg) was digested overnight at 37° C. with 10 units each of restriction enzymes EcoRI and BamHI (Pharmacia) in a final volume of 100 μl. The restriction fragments were separated by electrophoresis on a 0.8% agarose gel, 1× TBE. A piece of the gel corresponding to fragments between 3.5 kb and 2.5 kb in length was removed and DNA was purified out of the agarose gel using the QIAQuick gene extraction kit (QIAGEN) in a final volume of 30 μl.

The purified fragments were inserted by ligation between the EcoRI and BamHI restriction sites of the pBluescript II SK(+) vector (Stratagene). 1 μg of pBluescript SK(+) plasmid DNA was first digested with 5 units each of EcoRI and I restriction enzymes (Pharmacia) in 50 μl (37° C., 16 h) and subsequently purified from both enzymes using the QIAQuick gene extraction kit. The ligation was performed using 3 μl of purified genomic DNA fragments, 0.25 μg of digested pBluescript SK(+) DNA, 3 units of T4 DNA ligase (Pharmacia), 1 mM ATP in a final volume of 30 μl (1× One-Phor-All buffer PLUS, 16° C., 16 h). the ligation mixture was then dialysed on a VSWP 013 membrane (Millipore) against water for 20 min. 1 μl of this mixture was electroporated into 40 μl electrocompetent Escherichia coli DH10b cells (BRL-Gibco) according to the manufacturer's protocol. After electroporation, cells were plated on LB plates supplemented with 100 μg/ml ampicillin to select for the transformed cells.

The above-described library was screened progressively using PCR reactions on pools of transformants of decreasing sizes. The PCR reaction conditions were the same as described above with the exception that the template DNA was the plasmids from the pooled Excherichia coli transformants purified from 3 ml cultures with the High Pure Plasmid Isolation Kit (Boehringer Mannheim). A 0.3 kb fragment was amplified in one clone out of approximately 1000 clones analyzed. The plasmid (LMBP-39187) recovered from this clone (pPGXYNA) contained one EcoRI-BamHI insert of 3 kb length. A partial sequence of the pPGXYNA plasmid comprising the xylanolytic enzyme coding sequence was determined on both strands by primer walking using among others the oligonucleotide with the sequence SEQ ID NO 7 as primer.

SEQ ID NO 7: TAT TTC ACC TCT GGC AAG GGC T

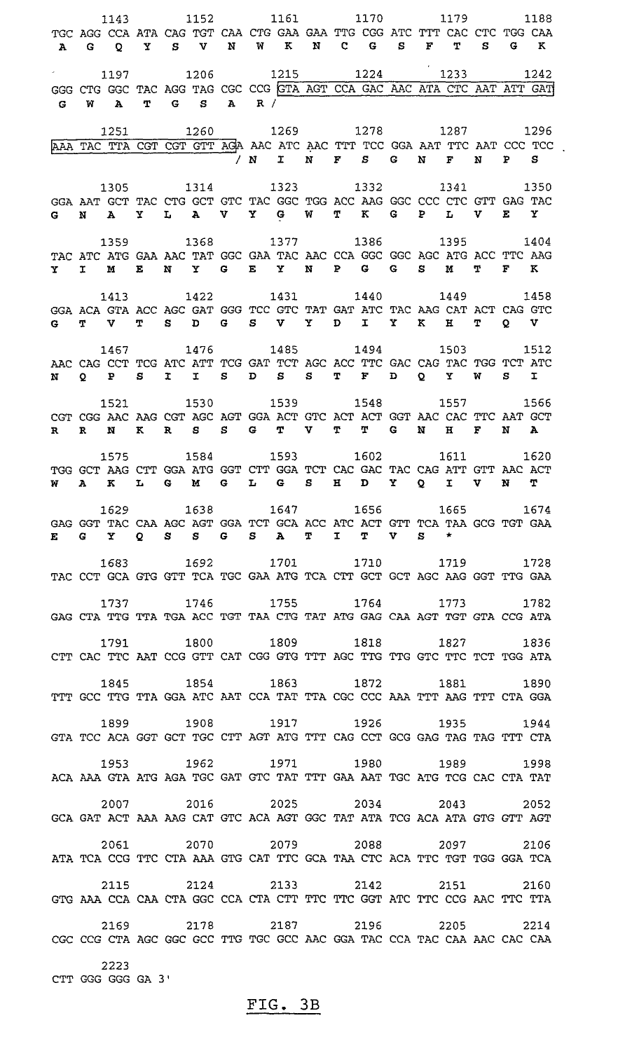

The nucleotide sequence SEQ ID NO 8 according to the invention codes for an amino acid sequence SEQ ID NO 11 and the localization of an intron was deduced from alignments of the Penicillium griseofulvum A160 sequence with the most homologous xylanase protein sequences obtained from a homology search in GENBANK with the BLASTP 2.0.8 software (Altschul et al., (1997) Nucl. Ac. Res., vol. 25, p. 3389.). This localization was confirmed by the presence of the putative lariat-formation internal sequence and with the definition of the consensus 5′ and 3′ splice-junction sequences (‘GT-AG’ rule). The sequences SEQ ID NO 9 and SEQ ID NO 10 are the sequences encoding the two exons of the enzyme with xylanolytic activity. The sequence SEQ ID NO 11 is the amino acid sequence of the Penicillium griseofulvum A160 enzyme. A signal sequence driving the secretion of the enzyme covers the first 27 amino acids of the sequence (FIG. 3).

### Example 4

### Expression of the Xylanolytic Enzyme Gene in Aspergillus oryzae

Construction of Expression Vectors

A DNA fragment covering the coding region as well as its terminator region was amplified by PCR. The first synthetic oligonucleotide SEQ ID NO 12 was chosen to contain the ATG codon corresponding to the first methionine of the coding region of the polypeptide gene as well as a recognition site for the restriction enzyme EcoRI. The second oligonucleotide SEQ ID NO 13 corresponded to the sequence located 250 bp downstream of the last codon and contained a XbaI restriction site.

| | |
|---|---|
| SEQ ID NO 12: | GGAATTCCATAATGGTCTCTTTCT |
| SEQ ID NO 13: | GCTCTAGAGCCACTTGTGACATGCT |

Both primers (40 pmoles) were used for a PCR reaction with approximately 40 ng of pPGXYNA plasmid DNA as template. The 100 μl PCR reaction also contained 2.5 units Pfu DNA polymerase (Stratagene) and 1 μg BSA in the following buffer: Tris-HCl pH 8.0 20 mM, KCl10 mM, MgCl₂ 2 mM, (NH4)₂SO₄ 6 mM and Triton X-100 0.1%. After denaturation of the DNA for 4 min at 94° C., 20 cycles of elongation were performed [30 s at 94° C., 30 s at 55° C. and 60 s at 72° C.] followed by an elongation step of 7 min at 72° C. The amplified DNA fragment was purified with the QIAQuick PCR purification kit (Qiagen) according to the manufacturer's protocol and recovered in a final volume of 50 μl. The extremities of the fragment were removed by digestion with the EcoRI and XbaI restriction enzymes (5 units of XbaI and 5 units of EcoRI enzymes (Pharmacia), 1× One-Phor-All buffer PLUS, final volume 60 μl, 37° C., overnight). The fragment was then purified with the QIAQuick gel extraction kit (Qiagen) after separation by electrophoresis on an agarose gel and recovered in 30 μl water.

The PCR DNA fragment was inserted between the EcoRI and XbaI restriction sites of the pBluescript II SK(+) vector (Stratagene). The vector was prepared as follows: 0.5 μg pBluescript SK(+) DNA was digested with 5 units EcoRI and 5 units XbaI restriction enzymes (Pharmacia) (final volume 20 μl, 2× One-Phor-All buffer PLUS, 37° C., overnight). After separation by electrophoresis in an agarose gel, it was purified with the QIAQuick gel extraction kit (Qiagen) and recovered in 30 μl water.

2 μl of PCR DNA fragment were ligated with this vector (1 μl) in the presence of ATP (1 mM), 1 unit of T4 DNA ligase (Pharmacia) and 1× One-Phor-All buffer PLUS (final volume 10 μl, 16° C., overnight). 1 μl of the ligation mixture was electroporated into electrocompetent Escherichia coli DH10b cells (BRL-Gibco) after dialysis against water. A clone was selected after analysis of a number of transformants plasmids by extraction, digestion with appropriate restriction enzymes and separation by electrophoresis on an agarose gel using standard procedures. The new plasmid was termed pPGXYNlE-X.

The promoter of the glyceraldehyde-3-P dehydrogenase gene from Aspergillus nidulans was cloned in front of the xylanolytic enzyme gene. This promoter allows a strong constitutive transcription of the genes located downstream of it (Punt et al., (1990), Gene, vol. 93, p. 101.; Punt et al., (1991), J. Biotechnol., vol. 17, p. 19.). The plasmid pFGPDGLAT2 contains this promoter between two restriction sites: EcoRI and NcoI. This promoter was inserted into the pBluescript II SK(+) plasmid between two EcoRI restriction sites to give the pSK-GPDp plasmid using standard procedures. This plasmid (1 μg) as well as PPGXYNlE-X (1 μg) were digested by the EcoRI restriction enzyme (5 units) in the presence of 1× One-Phor-All buffer PLUS (final volume 10 μl, 37° C., overnight). The DNA fragments of interest were then separated by electrophoresis on an agarose gel and purified with the QIAQuick gel extraction Kit (Qiagen) and collected in 30 μl water. The purified promoter DNA fragment (1 μl) was inserted by ligation between the EcoRI recognition sites of pPGXYNlE-X (1 μl) in the presence of ATP (1 mM), 1 unit of T4 DNA ligase (Pharmacia) and 1× One-Phor-All buffer PLUS (final volume 10 μl, 16° C., overnight). 1 μl of the ligation mixture was electroporated into electrocompetent Escherichia coli DH10b cells (BRL-Gibco) after dialysis against water. A clone was selected after analysis of a number of transformant plasmids by extraction, digestion with appropriate restriction enzymes and separation by electrophoresis on an agarose gel using standard procedures. The new plasmid was termed PGPDp-PGXYN1.

Transformation of Aspergillus oryzae

The strain Aspergillus oryzae MUCL 14492 was transformed by generating protoplasts according to the protocol described by Punt et al. (1992. Meth. Enzymol., vol. 216, p. 447.). The pGPDp-PGXYN1 plasmid was cotransformed with the p3SR2 plasmid that contains a selection marker used to recover transformants (the Aspergillus nidulans acetamidase gene—Hynes et al., (1983), Mol. Cell. Biol., vol. 3, p. 1430.). Transformants were selected on minimum medium plates containing acetamide as sole nitrogen source.

The strain Aspergillus oryzae MUCL 14492 was grown in 500 ml Aspergillus Minimum Liquid medium (Pontecorvo et al. (1992)) for 16 hours at 30° C. The culture was filtered through a Miracloth filter to collect the mycelium. The mycelium was washed with the Osm solution (CaCl₂ 0.27 M, NaCl 0.6 M) and then incubated with 20 ml solution Osm/g mycelium supplemented with 20 mg Novozym 234 (Sigma). After 1 hour at 30° C. with slow agitation (80 rpm), the protoplasts were formed and the suspension was placed on ice. The protoplasts were separated from intact mycelium by filtration through a sterile Miracloth filter and diluted with 1 volume STC1700 solution (sorbitol 1.2 M, Tris-HCl pH 7.5 10 mM, CaCl₂ 50 mM, NaCl 35 MM). The protoplasts were then collected by centrifugation at 2000 rpm for 10 min at 4° C. and washed twice with STC1700 solution. They were resuspended in 100 μl of STC1700 (10⁸ protoplasts/ml) in the presence of 3 μg p3SR2 plasmid DNA and 9 μg pGPDp-PGXYN1 plasmid DNA. After 20 min at 20° C., 250 μl, 250 μl and 850 μl PEG solution (PEG 4000 60%, Tris-HCl pH 7.5 10 mM and CaCl₂ 50 mM) were added successively and the suspension was further incubated for 20 min at 20° C. PEG treated protoplast suspensions were diluted by the addition of 10 ml STC1700 and centrifugated at 2000 rpm for 10 min at 4° C. The protoplasts were then resuspended in 200 μl STC1700 and plated onto Aspergillus Minimum Agar Medium and osmotically stabilized with 1.2 M sorbitol. To select the transformants, the nitrogen sources in the plates were replaced by 10 mM acetamide and 12 mM CsCl.

Analysis of Aspergillus oryzae Transformants

48 transformants were analyzed for the xylanolytic enzyme expression. They were grown in Aspergillus Minimum Liquid Medium supplemented with 3% sucrose as carbon source and 0.5% Bacto yeast extract (Difco). After 75 hours at 30° C. and 130 rpm, the supernatant of the cultures was assayed for xylanolytic activity. Ten of the transformants showed a significantly higher xylanolytic activity as compared to a control strain transformed only with the p3SR2 plasmid.

### Example 5

### Characterization of the Enzyme with Xylanolytic Activity from Penicillium griseofulvum A160

Purification of the Enzyme with Xylanolytic Activity Expressed in Aspoergillus oryzae

The enzyme with xylanolytic activity expressed in Aspergillus oryzae was purified in order to separate it from the traces of alpha-amylase present in the culture supernatants of the transformants. 10 ml of a culture supernatant from a selected transformant were diluted 3 times to reach a final concentration of 50 mM in sodium acetate pH 4.2.

This solution was located at 2 ml/min on a Pharmacia XK16/20 column filled with approximately 30 ml of the Bio-Rad Macro High S resin equilibrated in 50 mM sodium acetate pH 4.2. Proteins were eluted with a linear increasing NaCl gradient from 0 M to 0.6 M in 50 mM sodium acetate pH 4.2. Xylanase and amylase activities were determined in the eluted fractions. The amylase activity was recovered in the flow through fractions while the xylanase activity was eluted approximately at 0.1 M NaCl. The active fractions with xylanolytic activity were pooled and kept for further analysis.

Optimum pH and Temperature

The pH and temperature dependence of the activity of the xylanolytic enzyme secreted by one Aspergillus oryzae transformant was analyzed. The activity was measured in a citrate/phosphate buffer (0.1 M) at various pH (FIG. 4). The maximum activity was observed around 50° C. At this temperature, the optimum pH was about 5.0. These properties are similar to those of the enzyme with xylanolytic activity purified from the Penicillium griseofulvum A160.

### Example 6

### Baking Trials

Baking trials were performed to demonstrate the positive effect of the Aspergillus griseofulvum A160 xylanase in baking. The positive effect was evaluated by the increase in bread volume compared to a reference not containing the enzyme.

The xylanase was tested in Belgian hard rolls that are produced on a large scale every day in Belgium. The procedure described is well known to the craft baker and it is obvious to one skilled in the art that the same results may be obtained by using equipment from other suppliers.

The ingredients used are listed in Table 1 below:

TABLE 1

| Ingredients | RECIPE | RECIPE | RECIPE | RECIPE | RECIPE |
|---|---|---|---|---|---|
| (g) | 1 | 2 | 3 | 4 | 5 |
| Flour (Surbi- | 1500 | 1500 | 1500 | 1500 | 1500 |
| Molens van | | | | | |
| Deinze) | | | | | |
| Water | 915 | 915 | 915 | 915 | 915 |
| Fresh yeast | 90 | 90 | 90 | 90 | 90 |
| (Bruggeman- | | | | | |
| Belgium) | | | | | |
| Sodium | 30 | 30 | 30 | 30 | 30 |
| chloride | | | | | |
| Ascorbic | 0.12 | 0.12 | 0.12 | 0.12 | 0.12 |
| acid | | | | | |
| Multec Data | 3.5 | 3.5 | 3.5 | 3.5 | 3.5 |
| 2720S ™ | | | | | |
| Dextrose | 10 | 10 | 10 | 10 | 10 |
| Xylanase ™ | 0 | 23 | 35 | 52 | 70 |
| A160 | | | | | |
| (Megazyme | | | | | |
| units) | | | | | |

The ingredients were mixed for 2 min at low speed and 7 min at high speed in a Diosna SP24 mixer. The final dough temperature as well as the resting and proofing temperatures were 25° C. After resting for 15 min at 25° C., the dough was reworked manually and rested for another 10 min. Afterwards, 2 kg dough pieces were made up and proofed for 10 min. The 2 kg dough pieces were divided and made up using the Eberhardt Optimat. 66 gr round dough pieces were obtained. After another 5 min of resting time, the dough pieces were cut by pressing and submitted to a final proofing stage for 70 min.

The dough pieces were baked at 230° C. in a Miwe Condo™ oven with steam (Michael Wenz—Arnstein—Germany). The volume of 6 rolls was measured using the commonly used rapeseed displacement method.

The results are presented in Table 2 below:

TABLE 2

| Xylanase | Volume |
|---|---|
| units | (ml) |
| 0 | 2125 |
| 23 | 2475 |
| 35 | 2550 |
| 52 | 2675 |
| 70 | 2775 |

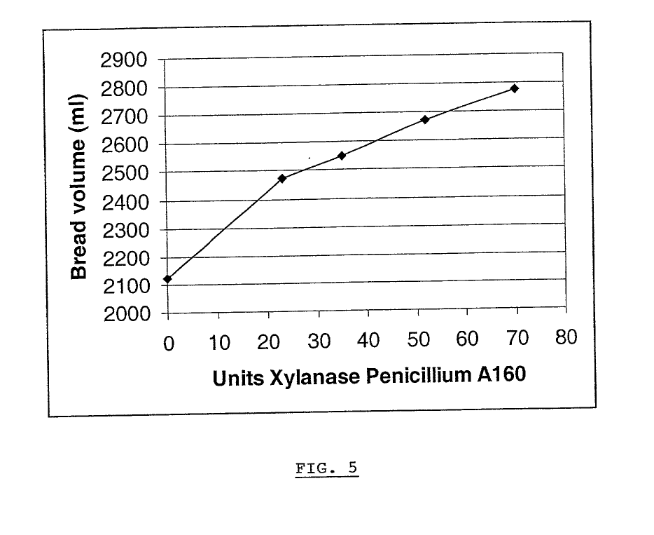

A graphical representation of the effect of the xylanase on bread volume is shown in FIG. 5.

### Example 7

### Effect of the Enzyme on the Flour Viscosity in the Presence of Water

Purified xylanase was used for the test. The enzyme was purified as described in Example 5. 100 g of wheat flour (Surbi, Molens van Deinze) were mixed manually with 117 ml water containing 25 xylanase units of the enzyme with xylanolytic activity from Penicillium griseofulvum A160. After 15 min at 36° C., the viscosity was measured (Programmable DV-II+Viscometer, Helipath system, Spindel F, Brookfield). The speed was maintained at 4 rpm and the viscosity value was measured after 10 s. The viscosity of a blank sample was obtained in the same way with untreated flour. The same experiment was also carried out with 10 units of the best performing enzyme available actually on the market (Aspergillus aculeatus xylanase available from Novo Nordisk (Shearzyme™ L)). Each experiment was performed in triplicate. The viscosity results presented in Table 3 below are expressed in centipoises.

TABLE 3

| | |
|---|---|
| Blank sample | 116.000 +/− 2000 |
| Penicillium griseofulvum enzyme | 65.034 +/− 5047 |
| Aspergillus aculeatus xylanase | 68.959 +/− 2253 |

Aspergillus aculeatus xylanase was shown to give better results than xylanases from Humicola insolens, Trichoderma reesei (Spezyme CP, Genencor) and another xylanase from Aspergillus aculeatus (xylanase I) (Patent application WO 94/21785). Christophersen et al. (Christophersen et al., (1997), Starch/Starke, vol. 49, p. 5.) also showed the better performance of Aspergillus aculeatus xylanase as compared to a xylanase from Thermomyces lanuginosus and two commercial hemicellulase cocktails sold for wheat separation.

The results presented above showed that the enzyme with xylanolytic activity from Penicillium griseofulvum A160 has the biggest capacity of reducing the viscosity of flour suspended in water.

### Example 8

### Wheat Separation

When mixed with water, the flour may be separated into a starch, a gluten, a sludge and a soluble fraction by centrifugation. A decrease of the sludge fraction leads to a better wheat separation. The performances of a pure xylanase can therefore be evaluated by measuring the decrease of the solid sludge fraction after centrifugation.

Such experiment has been carried out with the purified enzyme with xylanolytic activity from Penicillium griseofulvum A160 of Example 5 compared to Sherzyme™ L.

100 g of wheat flour (Surbi, Molens of Deinze) were mixed manually with 117 ml water containing different concentrations of enzyme. After 15 min at 35° C., the mixture was centrifugated for 10 min at 4000 g (Varifuge 3.0R, Heraeus Sepatech). The liquid phase was weighed.

Table 4 below shows the results of a typical experiment, by reporting the relative increase of the liquid phase induced by the presence of the xylanolytic enzyme. The enzyme from Penicillium griseofulvum A160 allowed to reach a higher liberation of liquid than Shearzyme™ L.

TABLE 4

| Enzyme | P. griseofulvum enzyme | Shearzyme ™ L |
|---|---|---|
| (units/test) | (%) | (%) |
| 0 | 100 | 100 |
| 3.125 | 110 | 127 |
| 6.25 | 129 | 130 |
| 12.5 | 134 | 134 |
| 25 | 166 | 137 |

The applicant has made a deposit of microorganism for the strain Penicillium griseofulvum Diercks A160 according to the invention under the deposit number MUCL 41920 on Dec. 13, 1999 at the BCCM/MUCL Culture Collection (Mycothèque de l'Université Catholique de Louvain, Place de la Croix du Sud 3, B-1348 LOUVAIN-LA-NEUVE, BELGIUM) and the deposit of the microorganism Escherichea coli DH10B (pPGXYNA) according to the invention on Dec. 13, 1999 under the deposit number LMBP 39987 at the Laboratorium voor Moleculaire Biologie BCCM/LMBP (K. L. Ledeganckstraat 35, B-9000 GENT, BELGIUM).

While the present invention has been described in some detail for purposes of clarity and understanding, one skilled in the art will appreciate that various changes in form and detail can be made without departing from the true scope of the invention. All figures, tables, and listings, as well as publications, referred to above, are hereby incorporated by reference.

What is claimed is:

1. An isolated or purified enzyme comprising SEQ ID NO:11, a homologue with more than 70% homology with the amino acid sequence SEQ ID NO:11 or a portion thereof.

2. The isolated or purified enzyme according to claim 1, having more than 80% homology with the amino acid sequence SEQ ID NO:11.

3. The isolated or purified enzyme according to claim 1, having more than 90% homology with the amino acid sequence SEQ ID NO:11.

4. The isolated or purified enzyme of claim 1 comprising the amino acid sequence of SEQ ID NO:1 or a portion thereof having a xylanolytic activity.

5. The isolated or purified enzyme according to claim 1, wherein said enzyme has optimum enzymatic activity at a pH between about 4.5 and about 7.0 and a temperature between about 35 and about 55° C.

6. An isolated or purified polynucleotide encoding the enzyme according to claim 1.

7. An isolated or purified polynucleotide more than 70% homologous to SEQ ID NO:8 or a portion thereof, wherein said polypeptide encodes a polypeptide having xylanolytic activity.

8. The isolated or purified polynucleotide according to claim 6, wherein said polynucleotide has more than 80% homology with SEQ ID NO:8.

9. The isolated or purified polynucleotide according to claim 6, wherein said polynucleotide has more than 90% homology with SEQ ID NO:8.

10. The isolated or purified polynucleotide of claim 7 comprising SEQ ID NO:8.

11. A recombinant polynucleotide comprising the polynucleotide of claim 6, operably linked to one or more regulatory sequence(s).

12. The recombinant polynucleotide of claim 11 wherein said one or more regulatory sequences(s) originate from microorganisms which are homologous to that the polynucleotide originated from.

13. A vector comprising the polynucleotide according to claim 6.

14. The vector of claim 13 comprising a plasmid incorporated in Escherichia coli and having the deposit number LMBP-39987.

15. A recombinant host cell transformed by the polynucleotide of claim 6.

16. The recombinant host cell according to claim 15 wherein said host cell is selected from the group consisting of archaea, bacteria and fungi.

17. The recombinant cell of claim 16 wherein said fungi is a yeast.

18. The recombinant host cell of claim 15 said host cell expresses the enzyme of SEQ ID NO:11, or a portion thereof intracellularly.

19. The recombinant host cell of claim 15 said host cell expresses the enzyme of SEQ ID NO:11, or a portion thereof extracellularly.

20. A solid support fixing an element selected from the group consisting of the cell according to claim 15, a cell extract of the cell according to claim 15 or the isolated or purified enzyme with xylanolytic activity expressed by the cell according to claim 15.

21. A method for the degradation of plant cell wall components comprising adding the enzyme of claim 1 to said plant cell wall components.

22. A method for the decomposition of plants and fruits comprising adding the enzyme of claim 1 to the preparation processes of fruit, legume juices, beer, paper, starch, gluten or vegetable oil.

23. A method for the decomposition of wastes comprising adding the enzyme of claim 1 to waste materials.

24. A method according to claim 23, wherein the waste materials are agricultural wastes or wastes from paper mills.

25. A method for increasing the volume of baked products comprising adding the enzyme of claim 1 to said baked products before baking.

26. A method for the separation of starch and gluten comprising adding the enzyme of claim 1 to a batter comprising starch and gluten.


SEQUENCE LISTING (14 sequences)

SEQ ID NO: 1 - protein, 26 aa, Penicillium griseofulvum
  VARIANT (1)...(26): Xaa = Any Amino Acid
DITQNERGTNNGYFYSFWTXGGGNVY

SEQ ID NO: 2 - DNA, 23 bp, Artificial Sequence
  degenerate primers based on N-terminal amino acid sequence
ggytayttytayaayttytggac

SEQ ID NO: 3 - DNA, 23 bp, Artificial Sequence
  degenerate primers based on N-terminal amino acid sequence
ggytaytaytaytcrttytggac

SEQ ID NO: 4 - DNA, 17 bp, Artificial Sequence
  hypothetical degenerate sequence
tcgacraygtagtaytc

SEQ ID NO: 5 - DNA, 102 bp, Penicillium griseofulvum
  misc_feature (1)...(102): n = A,T,C or G
cnagtacaacaacgnnaagnccggcnaatacagngtgnantggaagaactgcggntatttcacctctggcaagggctggannactggtagngcccggtaagt

SEQ ID NO: 6 - DNA, 87 bp, Penicillium griseofulvum
  misc_feature (1)...(87): n = A,T,C or G
cggcnaatacaagggtgtnantggaagaactgcggnnatttcacctcnggcaagggctggactactggtagtgcccggtaagtgcaa

SEQ ID NO: 7 - DNA, 22 bp, Artificial Sequence
  primer
tatttcacctctggcaagggct

SEQ ID NO: 8 - DNA, 2225 bp, Penicillium griseofulvum
  exon (962)...(1213)
  intron (1214)...(1261)
  exon (1263)...(1661)
  CDS (962)...(1213)
  CDS (1264)...(1662)
  misc_feature (1)...(2225): n = A,T,C or G
gaattctgctttgccaagnttcaacgcggagactcacagtcacattcttcgaatcttcttggcacgtgttcttgggtccttcgagaaatcatggatctggaaagttaaccagtaagccggttagaagacccggatcagcgacaaatagccggtagtaaattacttaatcgtatcgctagatctgatcatccgatagacaaacaaacaaacttaggctaccctagagatgaatcatgacagtagactattttaccaaggaatatttagaacaagcatacccctcactaattgggttgactatataaatacggttaaaagcatgggggactttcccaaggttgttcctgccaagctttgagatatacacccgttgatccatggatcaccgaggttgtccctgagctgtctcaagcttacaacaacttccaaggttctccaatgtcttatgagagctgataatcgaaataagatcaagtagccgatgtttccccggcttttaaactgcctgatcttgggtttagcctggccaagctacatccattatagccgtgatgaatttccccgcatttacacagccggtggctgaagtgtgcaacatgcttatttttacttgaagaagtttagccgactcaatagtttctacatgcttatttagctactaaaatctgattttagcctggttggatgatatagggatatagctgtcggtccgatggaccagtaatagttcatggacagtgaacatgacccgtgtttaacgtataattagtgcaattggaacagggcaaggggataaataggtcgttggctaaattcattcgagacatgtggaggactatgaaactgtttaaactcgccccacaccctccgtcaatataaaagaggtcttctccccaaggaatcatccatcacaaaacacactccaattcattcctcaattaccagcatctgacctttcataatggtctctttctcaagcctctttgtcgctgcatgcgccgctgtcagtgccctcgcgcttcccagtgacgtggaaaagcgcgacatcacccagaacgagcgaggaaccaacggcggctacttctactctttctggaccaacggtggcggcagtgtctcctacaacaacggcaatgcaggccaatacagtgtcaactggaagaattgcggatctttcacctctggcaagggctgggctacaggtagcgcccggtaagtccagacaacatactcaatattgataaatacttacgtcgtgttagaaacatcaacttttccggaaatttcaatccctccggaaatgcttacctggctgtctacggctggaccaagggccccctcgttgagtactacatcatggaaaactatggcgaatacaacccaggcggcagcatgaccttcaagggaacagtaaccagcgatgggtccgtctatgatatctacaagcatactcaggtcaaccagccttcgatcatttcggattctagcaccttcgaccagtactggtctatccgtcggaacaagcgtagcagtggaactgtcactactggtaaccacttcaatgcttgggctaagcttggaatgggtcttggatctcacgactaccagattgttaacactgagggttaccaaagcagtggatctgcaaccatcactgtttcataagcgtgtgaataccctgcagtggtttcatgcgaaatgtcacttgctgctagcaagggtttggaagagctattgttatgaacctgttaactgtatatggagcaaagttgtgtaccgatacttcacttcaatccggttcatcgggtgtttagcttgttggtcttctcttggatatttgccttgttaggaatcaatccatatttacgccccaaatttaagtttctaggagtatccacaggtgcttgccttagtatgtttcagcctgcggagtagtagtttctaacaaaagtaatgagatgcgatgtctattttgaaaattgcatgtcgcacctatatgcagatactaaaaagcatgtcacaagtggctatatatcgacaatagtggttagtatatcaccgttcctaaaagtgcatttcgcataactcacattctgttggggatcagtgaaaccacaactaggcccactacttttcttcggtatcttcccgaacttcttacgcccgctaagcggcgccttgtgcgccaacggatacccataccaaaaccaccaacttggggggga

SEQ ID NO: 9 - protein, 84 aa, Penicillium griseofulvum
MVSFSSLFVAACAAVSALALPSDVEKRDITQNERGTNGGYFYSFWTNGGGSVSYNNGNAGQYSVNWKNCGSFTSGKGWATGSAR

SEQ ID NO: 10 - protein, 133 aa, Penicillium griseofulvum
NINFSGNFNPSGNAYLAVYGWTKGPLVEYYIMENYGEYNPGGSMTFKGTVTSDGSVYDIYKHTQVNQPSIISDSSTFDQYWSIRRNKRSSGTVTTGNHFNAWAKLGMGLGSHDYQIVNTEGYQSSGSATITVS

SEQ ID NO: 11 - protein, 217 aa, Penicillium griseofulvum
MVSFSSLFVAACAAVSALALPSDVEKRDITQNERGTNGGYFYSFWTNGGGSVSYNNGNAGQYSVNWKNCGSFTSGKGWATGSARNINFSGNFNPSGNAYLAVYGWTKGPLVEYYIMENYGEYNPGGSMTFKGTVTSDGSVYDIYKHTQVNQPSIISDSSTFDQYWSIRRNKRSSGTVTTGNHFNAWAKLGMGLGSHDYQIVNTEGYQSSGSATITVS

SEQ ID NO: 12 - DNA, 24 bp, Artificial Sequence
  synthetic oligonucleotide
ggaattccataatggtctctttct

SEQ ID NO: 13 - DNA, 25 bp, Artificial Sequence
  synthetic oligonucleotide
gctctagagccacttgtgacatgct

SEQ ID NO: 14 - protein, 6 aa, Unknown
  conserved amino acid sequence from the family G xylanases
EYYIVD